(19) United States
(12) Patent Application Publication
(10) Pub. No.: US 2006/0234361 A1
(43) Pub. Date: Oct. 19, 2006

(54) Method for producing L-lactic acid

(75) Inventors: Dey-Chyi Sheu, Taipei City (TW); Kow-Jen Duan, Taipei City (TW); Ching-Yi Wu, Taipei City (TW); Mei-Ching Yu, Taipei City (TW)

Correspondence Address: BACON & THOMAS, PLLC, 625 SLATERS LANE, FOURTH FLOOR, ALEXANDRIA, VA, 22314, US

(73) Assignee: Tatung Company, Taipei City (TW)

(21) Appl. No.: 11/402,920

(22) Filed: Apr. 13, 2006

(30) Foreign Application Priority Data
Apr. 15, 2005 (TW) 094112044

(51) Int. Cl. C12P 7/56; C12N 1/16

(52) U.S. Cl. 435/139; 435/254.1

(57) ABSTRACT

A method for producing L-lactic acid is disclosed, which comprises the following steps: (a) culturing spores of Rhizopus oryzae in a medium containing a carbon source and a nitrogen source, forming dispersed mycelia, (b) seeding said mycelia into a fermentation medium containing ammonia; (c) starting the fermentation, meanwhile, the pH value is controlled in a range of 4-5 and the concentration of ammonia is in a range of 0.05-5 g/L; and (d) extracting the L-lactic acid form the fermentation medium, wherein the Rhizopus oryzae is Rhizopus oryzae ATCC 9363.

### BACKGROUND OF THE INVENTION

1. Field of the Invention

The present invention relates to a method of preparing L-lactic acid, more particularly, to a method of preparing L-lactic acid by Rhizopus oryzae.

2. Description of the Related Art

Pure L-lactic acid can be used to prepare biodegradable polymers, such as poly L-lactic acid (PLLA). The polymer is not only biodegradable but also biocompatible. PLLA can be applied in environmentally friendly packaging and other materials, or medical materials of medical implant for humans etc. Currently, the industrial process for preparing L-lactic acid by Rhizopus oryzae is mostly by means of submerged fermentation. However, during the fermentation, the mycelia of Rhizopus oryzae tend to twist together and form mycelia clumps easily. The mycelial clumps limit the substance transporting, such as nutrients, oxygen and generated products. Eventually, the mycelia clumps lower the producing rate, and the yield of L-lactic acid is thus reduced. The “yield” means the ratio of the produced L-lactic acid to the consumed glucose by mass.

In order to solve this problem, Kosakai, 1996, and Park, 1998, disclose methods with thread-like mineral support. The spores are seeded in the bioreactor directly. When the spores have sprouted and adhere to the mineral support, cotton-like mycelia flocs are thus formed. The yield of L-lactic acid by this method is 0.86-0.87. However, the mineral support used in this work might be asbestos, which is a dangerous chemical now banned worldwide. Furthermore, polyethylene oxide (PEO) is also a required component to be operated in coordination, for dispreading the mycelia well. Hence, these production methods disclosed above require long-term fermentation to obtained sufficient amounts of L-lactic acid. Besides, those methods disclosed above fail to teach large scale fermenting conditions, and therefore are inappropriate to be applied in the relevant industries.

Articles by Yin, 1998, and Bai, 2003 disclose producing L-lactic acid in a bioreactor by using mycelia pellets. The yield of L-lactic acid is only between 0.73-0.82 and the final concentration of lactic acid is less than other works.

Also, Miura S. et al. screened ammonia-tolerant mutants by treating the parent strain, Rhizopus sp. MK-96-1196, with NTG reagent. During the fermentation, the cultures of these strains are neutralized by ammonia water, thereby lactic acid is converted into ammonium lactate; and then the lactic acid is purified via n-butyl L-lactate without producing gypsum waste. However, the maximal concentration of lactic acid is 93 g/L, with a yield of 0.86, using 120 g/L of corn starch as substrate. Also, the method disclosed by Miura took 48 hours to complete the fermentation.

It is found that the main reason for forming mycelia clumps in cultures of Rhizopus oryzae is the depletion of the nitrogen source. The mycelial clumps are formed because the mycelia are cultured in an environment without sufficient nutrients (especially a nitrogen source). The immature cell walls of mycelia or the aged mycelia make the mycelia become adhesive and tend to aggregate together. According to the behavior described above, it is known that applying a nitrogen source to maintain appropriate nitrogen concentration in the culturing environment during fermentation is the key point to enhance the yield of lactic acid. Therefore, the mycelia flocs of Rhizopus oryzae can be dispread by controlling the concentration of nitrogen source. As a consequence, the production rate of L-lactic acid would be enhanced.

Therefore, it is desirable to provide an improved method to mitigate and/or obviate the aforementioned problems.

### SUMMARY OF THE INVENTION

The object of the present invention is to provide a method for preparing L-lactic acid by Rhizopus oryzae. The method of the present invention provides a fermentation medium, which is neutralized by calcium carbonate. Furthermore, the present method provides an appropriate environment for Rhizopus oryzae to ferment with sufficient nitrogen throught the fermentation process. Therefore, the mycelial flocs of Rhizopus oryzae are formed instead of mycelial clumps, hence, the yield of fermentation product—L-lactic acid—is increased.

The method for preparing L-lactic acid of the present invention comprises the steps of: (a) culturing spores of Rhizopus oryzae in a medium containing a carbon source and a nitrogen source, forming dispersed mycelia; (b) seeding the mycelia into a fermentation medium containing ammonia; (c) starting the fermentation, meanwhile, the pH value is controlled in a range of 4-5, and the concentration of ammonia is controlled in a range of 0.05-5 g/L; and (d) extracting the L-lactic acid from the fermentation medium, wherein, the Rhizopus oryzae is Rhizopus oryzae ATCC 9363.

According to the method of the present invention, the medium in step (a) is to provide an environment for forming dispersed mycelia from spores of Rhizopus oryzae. The composition of the medium used in the present invention can be any one or any combinations used in the art for dispersing mycelial flocs. Preferably, the medium used in the present invention contains a carbon source and a nitrogen source.

The carbon source can be any one or any combinations used in the art, but preferably, the carbon source is selected from a group consisting of glucose, sucrose, plant starch, fresh starch cassava starch, cornstarch, potato starch, grass starch, legume starch, wheat starch, rice bran, corn, wheat bran, barley, sweet potato, and molasses.

The nitrogen source can be any one or any combinations used in the art, but preferably, the nitrogen source is selected from a group consisting of yeast extract, soybean extract, protein hydrolysate, corn steep liquor, whey, urea, ammonium acetate, ammonium carbonate, and glutamic acid. The more preferable nitrogen source used in the present invention is glutamic acid.

Moreover, the composition of the medium is not limited, but preferably, the medium comprises 2-150 g/L of carbon source, and 0.1-20 g/L of nitrogen source. In order to maintain the growth of the cultured spores, some minerals are added to the culture media. Therefore, the medium in step (a) of the present invention contains some minerals, preferably, the mineral is selected from a group consisting of dipotassium hydrogen phosphate, potassium dihydrogen phosphate, magnesium sulfate, magnesium chloride, zinc sulfate, zinc nitrate and zinc chloride.

The fermentation medium in step (b) of the present invention method provides a suitable environment for producing L-lactic acid by Rhizopus oryzae. The composition of the fermentation medium can be any combinations of known medium ingredients, but preferably the reacting medium of the present invention comprises ammonia. The initial concentration of the ammonia is not limited, but preferably is in the range of 0.5-5 g/L. When the fermentation process starts in step (c), the concentration of ammonia in the fermentation medium is controlled in a range of 0.05-5 g/L, preferably is 0.05-0.5 g/L.

According to the method of the present invention, the nitrogen source of fermentation medium in step (b) can be any one of those conventionally used, but preferably is selected from the group consisting of ammonium sulfate, ammonium carbonate, ammonium phosphate, ammonium nitrate, ammonium chloride, ammonia, and urea. The more preferable nitrogen source is ammonium sulfate.

In the fermentation process of step (c), it is observed that the growth of the fungus, the consumption of medium ingredients and the production of lactic acid are strongly affected by the pH of the culture medium. Therefore, to keep the mycelia in normal growth in the present method, the pH value is preferably controlled in a range of 4-5. Moreover, the pH value in step (c) is controlled by any way used in the prior art, but preferably is controlled by adding calcium carbonate slurry.

Other objects, advantages, and novel features of the invention will become more apparent from the following detailed description when taken in conjunction with the accompanying figures.

### BRIEF DESCRIPTION OF THE FIGURES

FIG. 1 (A) shows the mycelia clumps of Rhizopus oryzae formed in the medium comprising ammonia sulfate.

FIG. 1 (B) shows the dispread mycelia floc of Rhizopus oryzae in the medium comprising glutamic acid;

FIG. 2 (A) shows the mycelia morphology of Rhizopus oryzae on the medium comprising ammonia sulfate;

FIG. 2 (B) shows the mycelia morphology of Rhizopus oryzae on the medium comprising glutamic acid;

FIG. 3 shows the time courses of glucose, lactic acid and dry-cell-weight (DCW) in the initial batch fermentation of Rhizopus oryzae ATCC 9363;

FIG. 4 shows the time courses of glucose, lactic acid and dry-cell-weight (DCW) in the repeated batch fermentation of Rhizopus oryzae ATCC 9363;

FIG. 5 shows the morphology of dispersed mycelial flocs of Rhizopus oryzae in a fermentation medium with a replenishment of NH₃; and

FIG. 6 shows the morphology of the coherent mycelial clumps of Rhizopus oryzae in a fermentation medium without further replenishment of NH₃.

### DETAILED DESCRIPTION OF THE PREFERRED EMBODIMENT

The L-lactic acid in the examples illustrated below of the present invention is produced by Rhizopus oryzae ATCC 9363 (BCRC 31837), which is purchased from the Bioresource Collection and Research Center, Hsinchu, Taiwan.

### Example 1

The ingredients of medium for the seed culture are as follows (g/L): glucose 50, glutamic acid 2, dipotassium phosphate (K₂HPO₄) 1, magnesium sulfate (MgSO₄.7H₂O) 0.5, zinc sulfate (ZnSO₄.7H₂O) 0.04.

The ingredients of the medium in the fermentation process are as follow (g/L): glucose 125, ammonia sulfate 3 (starting concentration), dipotassium phosphate (K₂HPO₄) 0.5, magnesium sulfate (MgSO₄.7H₂O) 0.25, zinc sulfate (ZnSO₄.7H₂O) 0.04.

The starting pH value is adjusted to 6.0 by adding sodium hydroxide in all culture media. During the fermentation for lactic acid production in the bioreactor, 40% CaCO₃ slurry is added to control the pH in a predetermined range. It is to be understood that the nitrogen source is replenished with ammonia sulfate if there is no further description in the following examples.

The ammonia concentration in the fermentation medium is assayed by an Orion ammonia electrode (Thermo Electron Co., USA). Rapid measurement of glucose concentration is carried out with a YSI 2700 biochemical analyzer (Yellow Spring Instruments Co., USA).

The concentration of lactic acid produced and the residual glucose is determined by HPLC on an ICSep ICE-ION-300 column (Transgenomic Inc., USA) under the following conditions: mobile phase, 0.0085 N H₂SO₄; flow rate, 0.4 mL/min; temperature, 30° C.; detector, Waters 410 differential refractometer. The retention times for glucose and lactic acid are 14.6 and 20.9 min, respectively. The measured concentrations of residual glucose and lactic acid are correlated to the initial volume.

The distinction between L-lactic acid and D-lactic acid is carried out by HPLC under the following conditions: column, Chiralpak MA(+), Daicel Chem. Ind. Ltd., Japan; mobile phase, 0.002 M CuSO₄; flow rate, 0.5 mL/min; temperature, 30° C. and detection at UV 254 nm. The retention times for D-lactic acid and L-lactic acid are 14.5 and 16.3 min, respectively.

The biomass of the fungus is represented as dry-cell-weight (DCW). Fifty mL of the liquid medium with mycelia is filtered through a glass-fiber filter (Advantec GC50). The mycelia is washed with 500 mL of water and dried to a constant weight at 60° C.

### Example 2

Spores of Rhizopus oryzae are dispread into an Erlenmeyer flask with a diameter of 14.5 cm, which contains a layer of potato dextrose agar in the bottom. The spores are cultured at 30° C. for 7-10 days. Fifty mL of aseptic 0.02% Tween-20 is used to suspend the grown spores by agitation. The concentration of spores is then calculated by hemacytometer under a microscope. Subsequently, the spores are inoculated into a 150 ml sprouting medium at a final concentration of 10⁶˜10⁷ spores per mL. The culture is incubated in a 30° C. shaker with an agitation rate of 150 rpm for 16-24 hours.

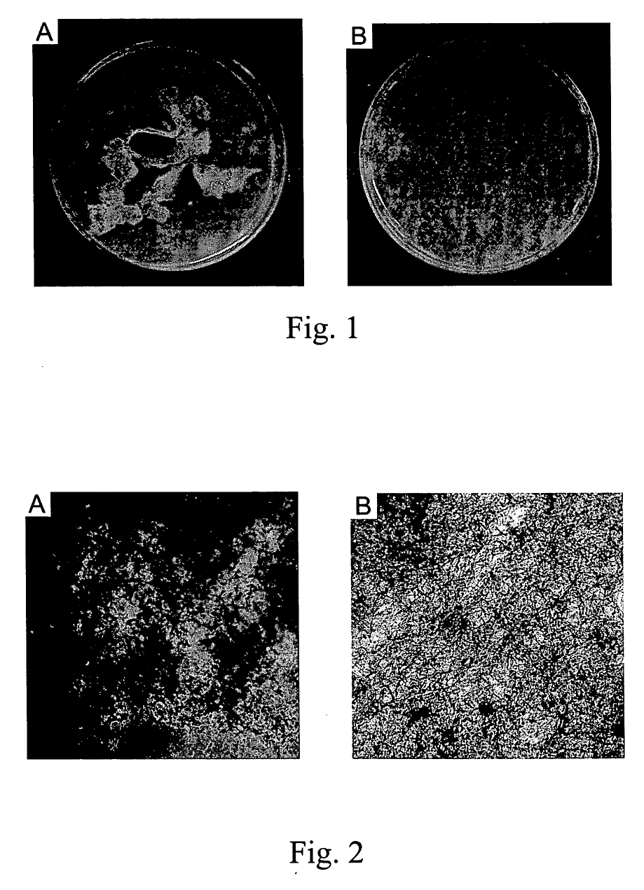

The above-mentioned shake flask cultures are cultivated for 18 hours. The morphology of the mycelia is observed by both naked eye and microscope. Most of the mycelia are aggregated, and large clumps are formed when glutamate in the medium is replaced by ammonium sulfate. The morphology observed by naked eye is shown in FIG. 1A, and the microstructure of the clump is shown in FIG. 1B. However, with glutamate as a nitrogen source, cotton-like floc morphology is induced and is shown in FIG. 2A, and the microstructure of the flocs is shown in FIG. 2B. According to the results described above, different nitrogen source has varied effect on hyphal elongation and branching, and the overall morphology—clump or floc—will determine the production rate of L-lactic acid. Using glutamate as a nitrogen source in shake flask culture is beneficial for Rhizopus oryzae to form floc morphology.

### Example 3

The production process, conditions, and the analysis of lactic acid in the present example are similar to the descriptions in example 1 and the preparation of seed culture is similar to that in example 2, where glutamate is used as a nitrogen source.

For lactic acid production, 300 ml of seed culture is inoculated into a 5-L stirred tank bioreactor containing 3 liter of fermentation medium. The fermentation is carried out under the following conditions: temperature 35° C., agitation rate 300 rpm, aeration rate 2 vvm. The pH of the culture is controlled in a predetermined range by automatic addition of 40% CaCO₃ slurry. A 25% (NH₄)₂SO₄ solution is added to the culture at 8-hour intervals in order that 0.5 g/L of ammonium concentration is achieved.

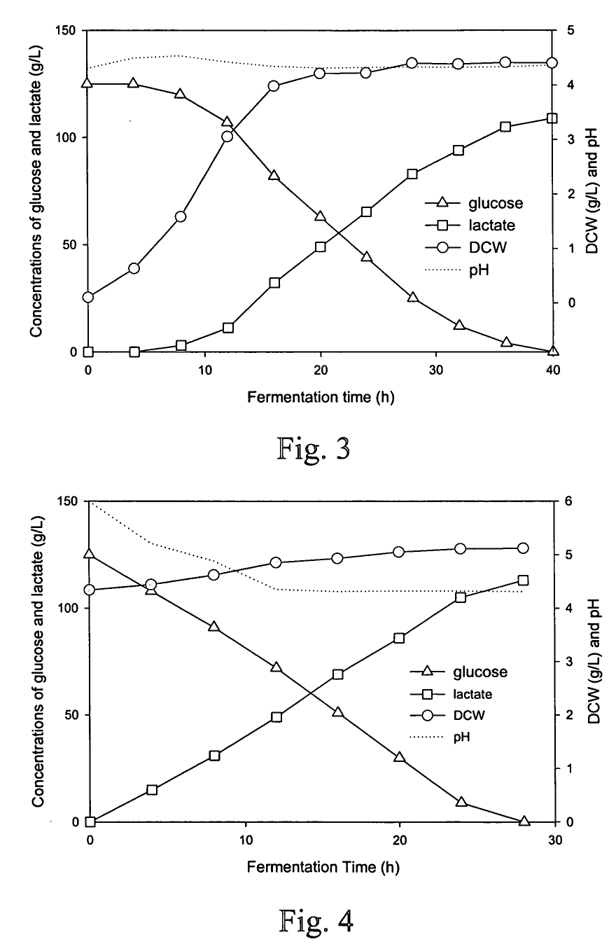

The time courses of glucose, lactate, dry-cell-weight (DCW) and pH value in the initial batch fermentation process are shown in FIG. 3. After 40 hours of fermentation, glucose is depleted completely and the final concentration of lactate is 109 g/L. The average production rate of lactic acid is 2.73 g/L per hour, and the yield of lactic acid is 0.87.

For the repeated batch production of lactic acid, the initial fermentation broth is filtered out using a glass tube with a sintered sparger head, which is connected to a peristaltic pump. The mycelia are washed with water for the following cycle of fermentation. And then 3 liter of the fermentation medium is supplied (g/L): ammonia sulfate 2, glucose 125, dipotassium phosphate 0.15, magnesium sulfate 0.25, zinc sulfate 0.04, and the pH is 6. The fermentation condition followed that of example 1. The time courses of glucose, lactate, DCW and pH value in this repeated batch fermentation process is shown in FIG. 4. After 28 hours of fermentation, the glucose is depleted completely and the final concentration of lactic acid is 113 g/L. The producing rate of lactic acid is 4.04 g/L per hour, and the yield of L-lactic acid is 0.90.

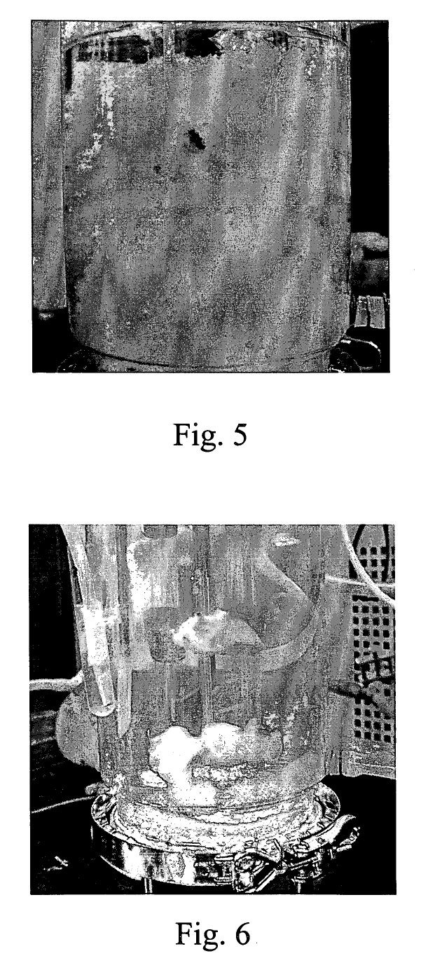

It is concluded that in the method for lactic acid production in the present example, as shown in FIG. 5, Rhizopus oryzae exhibits floc morphology, when the concentration of NH₃ is controlled at a predetermined level, and it is beneficial to the production of lactic acid. Otherwise, if the consumed NH₃ is not replenished, as shown in FIG. 6, the mycelia are aggregated to clumps.

Of the various techniques used currently, the present method for producing L-lactic acid, as illustrated in the examples above, provides a better yield and a higher production rate than other techniques in which mycelial pellets or immobilized cells are used. Furthermore, the present method is readily to be conducted in the industrial process for the production of L-lactic acid.

Although the present invention has been explained in relation to its preferred embodiment, it is to be understood that many other possible modifications and variations can be made without departing from the spirit and scope of the invention as hereinafter claimed.

What is claimed is:

1. A method for producing L-lactic acid comprising the steps of:
(a) culturing spores of Rhizopus oryzae in a medium containing carbon source and nitrogen source, forming dispersed mycelia;
(b) seeding the mycelia into a fermentation medium containing ammonia;
(c) starting the fermentation, meanwhile, the pH value is controlled in a range of 4-5, and the concentration of ammonia is in a range of 0.05-5 g/L; and
(d) extracting the L-lactic acid from the fermentation medium, wherein, the Rhizopus oryzae is Rhizopus oryzae ATCC 9363.

2. The method as claimed in claim 1, wherein said carbon source of step (a) is selected from a group consisting of: glucose, sucrose, plant starch, fresh starch, cassava starch, corn starch, potato starch, grass starch, legume starch, wheat starch, rice bran, corn, wheat bran, barley, sweet potato and molasses.

3. The method as claimed in claim 1, wherein said nitrogen source of step (a) is selected from a group consisting of: yeast extract, soybean extract, protein hydrolysate, corn steep liquor, whey, urea, ammonium acetate, ammonium carbonate, and glutamic acid.

4. The method as claimed in claim 1, wherein said medium comprises carbon source of 2-150 g/L, nitrogen source of 0.1-20 g/L.

5. The method as claimed in claim 1, wherein said fermentation medium of step (b) comprises ammonia of 0.05-5 g/L.

6. The method as claimed in claim 1, wherein said medium of step (a) further comprises at least one mineral.

7. The method as claimed in claim 6, wherein said mineral is selected from a group consisting of: dipotassium hydrogen phosphate, potassium dihydrogen phosphate, magnesium sulfate, zinc sulfate, ammonium sulfate, and the combination thereof.

8. The method as claimed in claim 1, wherein said concentration of ammonia of step (c) is controlled in a range of 0.05-5 g/L.

9. The method as claimed in claim 1, wherein said ammonia is selected from a group consisting of: ammonium sulfate, ammonium carbonate, ammonium phosphate, ammonium nitrate, ammonium chloride, ammonia, and urea.

10. The method as claimed in claim 1, wherein said pH of step (c) is controlled in a range of 4-5.

11. The method as claimed in claim 1, wherein said pH of step (c) is controlled by adding calcium carbonate.

12. The method as claimed in claim 1, wherein said Rhizopus oryzae is cultured at a temperature ranged from 25° C. to 40° C.
